(19) United States
(12) Patent Application Publication
(10) Pub. No.: US 2006/0134022 A1
(43) Pub. Date: Jun. 22, 2006

(54) Salivary stimulatory compositions and method of use thereof

(76) Inventors: Leonard Mackles, New York, NY (US); William S. Bess, Edison, NJ (US)

Correspondence Address: OMRI M. BEHR, 325 PIERSON AVENUE, EDISON, NJ, 08837-3123, US

(21) Appl. No.: 11/256,365

(22) Filed: Oct. 21, 2005

Related U.S. Application Data
(63) Continuation-in-part of application No. 11/019,486, filed on Dec. 22, 2004, now abandoned.

(51) Int. Cl. A61K 8/22

(52) U.S. Cl. 424/53

(57) ABSTRACT

Effective stimulation of oral salivary activity in the oral cavity is achieved by the introduction thereinto of a warm aqueous solution, containing 0.5-15 wt % of an inorganic cation salt of a sulfate, bisulfate pyrosulfate or mixture thereof, having a temperature of at least 103° F. (39.4° C.).

### RELATED APPLICATIONS

This application is a continuation in part of our application Ser. No. 11/019,486 Filed Dec. 22, 2004

### FIELD OF USE

Salivary stimulation

### BACKGROUND OF THE INVENTION

It has long been recognized that the presence of adequate amounts of saliva in the oral cavity is desirable. The stimulation of saliva production for counteracting dry mouth and xerostomia has heretofore been achieved by numerous means. These may be topical or interventional, that is to say by acupuncture or surgery. In addition to the foregoing it has been postulated that the mere increase in saliva flow acts to reduce caries (Edgar et al Adv. Dent. Res. 1994July; 8(2): 239-45, and Caries Res. 2004, 38, 241-246).

Topical modes include chewing gum, with or without physiologically active ingredients, candies, acid tasting hard candies, and various mouth washes. Nevertheless, there has existed a need for an orally acceptable and pleasant rinse which rapidly produces a high level of salivary stimulation which persists for a reasonable time after the bulk of the rinse has been ejected from the mouth.

### SUMMARY OF THE INVENTION

There is provided a method of stimulating salivary activity in the oral cavity comprising the sequential steps of reacting an aqueous solution of between 1 and 30 wt % of a sulfite, bisulfite, metabisulfite, or mixture thereof, with an orally acceptable oxidizing agent or mixture of agents sufficient to oxidize said solution to provide a warm solution of 0.5-15 wt % of the corresponding sulfate, bisulfate, pyrosulfate or mixture thereof, having a temperature of at least 103° F. (39.4° C.) and promptly introducing said warm solution into the oral cavity. Suitably thereafter the solution is ejected from the mouth. The orally acceptable oxidizing agent is a peroxide, or mixture of peroxides suitably the peroxide is calcium peroxide, sodium peroxide, urea peroxide, hydrogen peroxide or mixtures thereof. Other orally acceptable oxidizing agents include cationic metal salts of a percarbonate, perborate, persulfate, or mixture thereof. It is particularly preferred if the orally acceptable oxidizing agent is hydrogen peroxide.

We have found that effective stimulation of oral salivary activity in the oral cavity is achieved by the introduction thereinto of an aqueous solution containing 0.5-15 wt % of an inorganic cation salt of a sulfate, bisulfate, pyrosulfate or mixture thereof, which has a temperature of at least 103° F. (39.4° C.).

Suitably the amount of salt is between about 1.5 and about 10 wt %, preferably between about 2 and about 6 wt %. Any cation that is physiologically or taste acceptable may be employed. It has been found desirable to utilize sodium, potassium, ammonium, magnesium, calcium, aluminum, zinc, iron or mixture thereof as the cation.

The saliva stimulation effectiveness of the solution depends on raising its temperature before use. This may be done by conventional external heating means. Alternatively the sulfate, bisulfate or pyrosulfate may be produced in situ by utilizing a solution of the corresponding sulfite, bisulfite or metabisulfite and adding a solution of an orally acceptable peroxide thereto. The resulting reaction produces excess heat, thus raising the solution temperature. While the amount of peroxide added is not critical, it is desirable to use at least an amount substantially equivalent to the stoichiometric amount need to oxidize the salts in their reduced forms to the higher oxidation state.

### EXAMPLES

### Example 1

### Self Warming Mouthwash Temperature Increase vs. Sulfite Concentration

Note: Temperatures are in Fahrenheit Degrees and are approximate (plus/minus 2 degrees).

Sulfite Concentrations listed below are final concentration in the mouthwash. Since the final mouthwash is formed from equal amounts of 2 separate phases, the sulfite concentration in the sulfite containing phase is double that in the final mouthwash.

| Sulfite - Concentration | | Initial Temp. | Final Temp. | Temp. Increase |
|---|---|---|---|---|
| Sodium Sulfite | 1% | 87.9 | 98.1 | 10.2 |
| | 2% | 89.3 | 110.7 | 21.4 |
| | 4% | 90.5 | 133.5 | 43.0 |
| Sodium | 1% | 85.2 | 97.3 | 12.1 |
| Metabisulfite | 2% | 86.0 | 113.2 | 27.2 |
| | 4% | 86.1 | 142.0 | 55.9 |
| Combined | 1% + 1% | 85.6 | 109.2 | 23.6 |
| Sulfite & | 2% + 2% | 86.2 | 136.0 | 49.8 |
| Metabisulfite | | | | |

### Example 2

### Bisulfite Composition Which has No Excess Peroxide

Temperature increase=55° F.

| Ingredient | % by Weight |
|---|---|
| Phase A: | |
| Ethanol | 10.00 |
| Flavor Oil | 0.20 |
| Magnesium Lauryl Sulfate | 0.27 |
| Sucralose (1% Aq. Sol'n) | 15.00 |
| Water | 61.53 |
| Sodium Bisulfite | 8.00 |
| Tromethamine | 5.00 |
| Phase A Total | 100.00 |
| Phase B: | |
| Hydrogen Peroxide (50% Aq. Sol'n) | 5.23 |
| Water | 94.77 |
| Phase B Total | 100.00 |

Phase A: Combine Ethanol Flavor Oil and Magnesium Lauryl Sulfate. Mix until clear. While mixing, add the Sucralose, water, Sodium Bisulfite and Tromethamine. Mix until a clear solution is obtained.

Phase B: Combine the Hydrogen Peroxide and water. Mix until uniform

Combine equal amounts of Phase A and Phase B, Just prior to use, to form the final mouth rinse

In accordance with the above formulation, but where in place of bisulfite there is utilized metabisulfite, or sulfite, a similar solution is obtained.

In accordance with the above formulation, but where in place of sodium the cation utilized is potassium, ammonium, magnesium, calcium, aluminum, zinc, iron or mixture thereof a similar formulation is obtained.

Similarly in place of hydrogen peroxide, there may be utilized urea peroxide or mixtures thereof with hydrogen peroxide.

The formulation is utilized by introducing a comfortable amount, suitably about 10 ml, into the mouth agitating it for about 30 seconds and ejecting it. Increased saliva flow is immediately noted.

### Example 3

### Mixed Sulfite/Metabisulfite Composition Which has 3% Excess Peroxide to Provide Whitening and Germ Kill in Addition to Saliva Stimulation

Mix equal amounts of Phase A & B just prior to introduction into the oral cavity. Temperature Increase=50 degrees F.

| Ingredient | % by Weight |
|---|---|
| Phase A | |
| Flavor | 0.20 |
| Alcohol, 95% Grain | 10.00 |
| Magnesium Lauryl Sulfate, 27% | 1.00 |
| Sucralose 1% Sol'n | 15.00 |
| Water | 45.80 |
| Sodium Metabisulfite | 4.00 |
| Sodium Sulfite | 4.00 |
| Tromethamine | 5.00 |
| Sorbitol, 70% | 15.00 |
| Total | 100.00 |
| Phase B | |
| Hydrogen Peroxide, 50% | 17.10 |
| Water | 82.90 |
| Total | 100.00 |

### Example 4

### Mixed Sulfite/Metabisulfite Composition Which has 3% Excess Peroxide to Provide Whitening and Germ Kill in Addition to Saliva Stimulation

Mix equal amounts of Phase A & B just prior to introduction into the oral cavity.

Temperature Increase=24 degrees F.

| Ingredient | % by Weight |
|---|---|
| Phase A | |
| Flavor | 0.20 |
| Alcohol, 95% Grain | 10.00 |
| Magnesium Lauryl Sulfate, 27% | 1.00 |
| Sucralose 1% Sol'n | 15.00 |
| Water | 51.80 |
| Sodium Metabisulfite | 2.00 |
| Sodium Sulfite | 2.00 |
| Tromethamine | 3.00 |
| Sorbitol, 70% | 15.00 |
| Total | 100.00 |
| Phase B | |
| Hydrogen Peroxide, 50% | 14.51 |
| Water | 85.49 |
| Total | 100.00 |

### Example 5

### Metabisulfite Composition Which has 3% Excess Peroxide to Provide Whitening and Germ Kill in Addition to Saliva Stimulation

Mix equal amounts of Phase A & B just prior to introduction into the oral cavity.

Temperature Increase=56 degrees F.

| Ingredient | % by Weight |
|---|---|
| Phase A | |
| Flavor | 0.20 |
| Alcohol, 95% Grain | 10.00 |
| Magnesium Lauryl Sulfate, 27% | 1.00 |
| Sucralose 1% Sol'n | 15.00 |
| Water | 38.80 |
| Sodium Metabisulfite | 8.00 |
| Tromethamine | 12.00 |
| Sorbitol, 70% | 15.00 |
| Total | 100.00 |
| Phase B | |
| Hydrogen Peroxide, 50% | 17.72 |
| Water | 82.28 |
| Total | 100.00 |

### Example 6

### Sulfite Composition Which has No Excess Peroxide. PROVIDES Saliva Stimulation

Mix equal amounts of Phase A & B just prior to introduction into the oral cavity.

Temperature Increase=43.0 degrees F.

| Ingredient | % by Weight |
|---|---|
| Phase A | |
| Flavor | 0.20 |
| Alcohol, 95% Grain | 10.00 |
| Magnesium Lauryl Sulfate, 27% | 1.00 |
| Sucralose 1% Sol'n | 15.00 |
| Water | 50.80 |
| Sodium Sulfite | 8.00 |
| Sorbitol, 70% | 15.00 |
| Total | 100.00 |
| Phase B | |
| Hydrogen Peroxide, 50% | 4.31 |
| Water | 95.69 |
| Total | 100.00 |

### Example 7

### Mixed Sodium/Potassium Sulfite Composition Which has No Excess Peroxide. Provides Saliva Stimulation.

Mix equal amounts of Phase A & B just prior to introduction into the oral cavity.

Temperature Increase=43.0 degrees F.

| Ingredient | % by Weight |
|---|---|
| Phase A | |
| Flavor | 0.20 |
| Alcohol, 95% Grain | 10.00 |
| Magnesium Lauryl Sulfate, 27% | 1.00 |
| Sucralose 1% Sol'n | 15.00 |
| Water | 49.80 |
| Sodium Sulfite | 4.00 |
| Potassium Sulfite | 5.00 |
| Sorbitol, 70% | 15.00 |
| Total | 100.00 |
| Phase B | |
| Hydrogen Peroxide, 50% | 4.31 |
| Water | 95.69 |
| Total | 100.00 |

### Example 9

### Mixed Sodium Sulfite/Potassium Metabisulfite Composition Which has 3% Excess Peroxide to Provide Whitening and Germ Kill in Addition to Saliva Stimulation

Mix equal amounts of Phase A & B just prior to introduction into the oral cavity.

Temperature Increase=50 degrees F.

| Ingredient | % by Weight |
|---|---|
| Phase A | |
| Flavor | 0.20 |
| Alcohol, 95% Grain | 10.00 |
| Magnesium Lauryl Sulfate, 27% | 1.00 |
| Sucralose 1% Sol'n | 15.00 |
| Water | 45.12 |
| Potassium Metabisulfite | 4.68 |
| Sodium Sulfite | 4.00 |
| Tromethamine | 5.00 |
| Sorbitol, 70% | 15.00 |
| Total | 100.00 |
| Phase B | |
| Hydrogen Peroxide, 50% | 17.10 |
| Water | 82.90 |
| Total | 100.00 |

We claim:

1. A method of stimulating salivary activity in the oral cavity comprising the sequential steps of
a) reacting an aqueous solution of between 1 and 30 wt % a sulfite, bisulfite, metabisulfite, or mixture thereof, with
b) an orally acceptable oxidizing agent or mixture of agents to oxidize said solution to provide a warm solution containing 0.5-15 wt % of the corresponding sulfate, bisulfate, pyrosulfate or mixture thereof, having a temperature of at least 103° F. and
c) promptly introducing said warm solution into the oral cavity

2. The method of claim 1 comprising the further step of ejecting said solution from the mouth.

3. The method of claim 1 where the orally acceptable oxidizing agent is a peroxide, or mixture of peroxides.

4. The method of claim 3 where the peroxide is calcium peroxide, sodium peroxide, urea peroxide, hydrogen peroxide or mixtures thereof.

5. The method in claim 1 where the orally acceptable oxidizing agent is a cationic metal salt of a percarbonate, perborate, persulfate, or mixture thereof.

6. The method in claim 1 where the orally acceptable oxidizing agent is a mixture of at least one peroxide and at least one compound selected from cationic metal salts of a percarbonate, perborate, or persulfate,

7. The method of claim 1 wherein the amount of salt is between about 1.5 and about 10 wt %.

8. The method of claim 7 wherein the amount of salt is between about 2 and about 6 wt %.

9. The method of claim 1 where the cation is selected from the group consisting of sodium, potassium, ammonium, magnesium, calcium, aluminum, zinc, iron or mixture thereof.

10. The method of claim 7 where the cation is selected from the group consisting of sodium, potassium, ammonium, magnesium, calcium, aluminum, zinc, iron or mixture thereof.

11. The method of claim 8 where the cation is selected from the group consisting of sodium, potassium, ammonium, magnesium, calcium, aluminum, zinc, iron or mixture thereof.
